(19) United States
(12) Patent Application Publication
(10) Pub. No.: US 2025/0197794 A1
(43) Pub. Date: Jun. 19, 2025

(54) MEMBRANE RUPTURE COMPOSITIONS AND METHODS OF MAKING AND USING SAME

(71) Applicant: Avantor Performance Materials, LLC, Radnor, PA (US)

(72) Inventors: Arvind Srivastava, Stewartsville, NJ (US); Nandkumar Deorkar, Cedar Knolls, NJ (US); Lori Fortin, Bethlehem, PA (US); Jungmin Oh, Somerville, NJ (US); Courtney O'Dell, Denville, NJ (US); Pranav S. Vengsarkar, Sumerville, NJ (US)

(73) Assignee: Avantor Performance Materials, LLC, Radnor, PA (US)

(21) Appl. No.: 18/031,517

(22) PCT Filed: Oct. 14, 2021
(86) PCT No.: PCT/US2021/055002

§ 371 Date: Apr. 12, 2023

Related U.S. Application Data
(60) Provisional application No. 63/091,718, filed on Oct. 14, 2020.

(51) Int. Cl. C12N 1/06; C12N 15/86

(52) U.S. Cl. CPC C12N 1/06; C12N 15/86; C12N 2750/14143; C12N 2750/14151; C12N 2750/14163

(57) ABSTRACT

The present invention provides a membrane rupture solution comprising: one or more purified non-ionic detergents, wherein at least one of the detergents has a surface activity property that is suitable for viral vector/protein stabilization against shear stress, and, optionally, a scavenger.

### CROSS-REFERENCE TO RELATED APPLICATIONS

This application claims the benefit of International Application No. PCT/US2021/055002, filed Oct. 14, 2021, which claims priority to the U.S. Provisional Application No. 63/091,718, filed Oct. 14, 2020, the disclosures of which are incorporated herein by reference in their entireties.

### FIELD OF THE INVENTION

The invention relates to cell membrane rupture compositions (e.g., solutions), and methods for preparing and using same. The membrane rupture solution comprises one or more non-ionic detergents along with additives, wherein at least one of the detergents has a surface activity property that is critical for cell, viral vector, and protein stabilization against shear stress.

### BACKGROUND OF THE INVENTION

In general, most cells have an outer membrane. The function of this membrane is to protect, organize, and regulate permeation into the cell. With a few exceptions, cell membranes consist of glycerophospholipids, proteins, polysaccharides, glycocalyx and proteoglycans. Cell lysis refers to rupturing of the cell membrane and subsequent breaking down of the cells. Various techniques such as enzymatic, osmotic, freeze-thaw and mechanical pressure are utilized for breaking the outer membrane of cells to release intracellular materials such as DNA, RNA, protein, or viral vectors. Some of these techniques can damage/denature proteins, DNA, and other constituents of interest. As such, there is a need for a cell lysis reagent that breaks down the cell membrane and protects the material of interest from damage during the potentially harsh manufacturing process. Cell lysis is also important for the molecular diagnostics of pathogens, immunoassays for point of care diagnostics, downstream processes such as protein purification for studying protein function and structure, downstream purification of viral vectors, cancer diagnostics, drug screening, mRNA transcriptome, and determination and analysis of the composition of specific proteins, lipids, and nucleic acids individually or as complexes.

There are several chemical or mechanical methods for cell lysis [2]. The rudimentary mechanical technique to liberate recombinant adeno-associated virus (rAAV) vectors from cells is freeze/thaw cycling followed by a low-speed centrifugation clarification step. However, this technique is not appropriate for large scale purification of rAAVs because it is difficult to scale accordingly. Mechanical homogenization is another lysis method wherein cells in media are forced through an orifice using high pressure. Disruption of the membrane occurs due to the high shear force as the cell is subjected to compression while entering the orifice and as it is expanded upon discharge. Although this method is scalable, it has a disadvantage of product loss due to shear stress-induced aggregation and precipitation.

Cell lysis by SDS works only at alkaline pH [3], which is not an optimal solution condition for viral particle stability as well other intracellular constituents [4]. In the SDS cell lysis process, viral vectors are released from host cells in harsh solution conditions, resulting in a significant loss of viral vectors due to processing.

The cell lysis process also releases nucleic acid impurities, the removal of which requires a separate unit operation. Current lysis methods do not prevent product loss due to shear stress, nor do they remove process related impurities resulting from the cell lysis process. Any solution that can combine both cell lysis and a process for DNA removal, while also preventing loss due to shear stress, will be of value to viral vector manufacturing.

Viral inactivation is a process where the surface properties of viruses become altered so that the virus is no longer active, or unable to infect. Drug manufacturers are required to ensure the viral safety of biological therapeutic products. Viral contamination is a common threat to all animal- and human-derived biopharmaceuticals. Viral contaminants can come from cell lines (e.g., endogenous retroviruses) or from adventitious (e.g., mycoplasma) introduction during drug manufacturing.

For viral inactivation, a drug manufacturer applies a variety of technologies to remove or inactivate virus. These include low pH inactivation, viral filtration, and chemical inactivation. No single strategy can ensure the safety of all biological products. However, a solvent/detergent treatment is the method of choice to inactivate lipid-coat enveloped viruses because it is robust and easy to scale-up.

Traditionally, various detergents such as Octoxynol-9 (also sold as Triton X-100,) sodium deoxycholate, and sodium dodecyl sulfate have been used as primary detergents for cell lysis [5]. Among them Triton X-100 has been the preferred detergent for viral vector purification processes [5] and inactivation of enveloped viruses due to its superior performance.

Research has shown that Triton X-100 causes acute oral toxicity, eye damage, skin irritation, and chronic aquatic toxicity [6]. As a result, the detergent has been placed on the “substance of very high concern list” in December 2016 by the European Chemicals Agency under REACH regulations [6]. REACH, which stands for Registration, Evaluation, Authorization and Restriction of Chemicals, is a European Union regulation enacted in December 2006 to address chemical safety on both human health and the environment. The detergent has been banned for use in Europe since January 2021 [7], and other regions of the globe could follow or suffer in Europe's wake.

Therefore there is a clear need for a safe and effective membrane rupture solution for cell lysis and viral inactivation that not only lyses the cells and releases intracellular materials from the host cell, but also protects the material of interest from damage during processing conditions. Desirably, the membrane rupture solution should also be able to isolate product without impacting it and remove process related impurities that are generated during the cell lysis process. The membrane rupture solution of this invention differs from current compositions and has shown surprisingly rapid and efficient lysis, while at the same time protecting the viral vector from damage due to shear stress. Furthermore, it removes product and process related impurities such as empty capsids, cell debris and cellular DNA.

### SUMMARY OF THE INVENTION

The instant invention provides non-ionic detergent mixtures which surprisingly protect cellular constituents of interest (e.g., viral vectors and proteins) while enabling quantitative cell lysis. The membrane rupture solutions of the instant invention disrupt the outer boundary of cell membranes, protect the material of interest from damage during the potentially harsh manufacturing process and separate the material of interest from product and process related impurities. There are two major applications of membrane rupture solutions: cell lysis and viral inactivation. Viruses are divided into two groups: enveloped viruses, the first group, are surrounded by an outer lipid membrane; non-enveloped viruses, the second group, lack this lipid membrane. Membrane rupture solutions inactivate enveloped viruses. The mechanisms of cell lysis and the inactivation of enveloped viruses are the same. Both cell membranes and enveloped viruses contain a lipid bilayer, which is made of hydrophobic and hydrophilic molecules. Membrane rupture solutions disrupt the lipid-lipid, lipid-protein and protein-protein interactions [2]. Therefore, any solution that can lyse the cells can also inactivate enveloped viruses.

The detergents of the instant membrane rupture solutions are benign, generate low foam, are environmentally compatible, and more specifically, meet the OECD guideline for readily biodegradable material according to OECD 301. The instant membrane rupture solutions, and methods of cell rupture, where the detergent is environmentally friendly, can protect the product of interest in the lysate (and potentially remove product and process related impurities in the case of cell lysis) or solution (in the case of viral inactivation) at close to physiological conditions.

In one aspect, the invention includes membrane rupture compositions (i.e., solutions) useful for cell lysis and viral inactivation. In one embodiment, the solution comprises one or more purified non-ionic detergents, wherein at least one of the detergents has a surface activity property that is suitable for viral vector/protein stabilization against shear stress (Table 1). Examples of suitable detergents include alcohol ethoxylates (e.g., TDA 9, Tergitol 15-s-9), Sorbiton ethoxylate (e.g., Polysorbate 20, Polysorbate 80), and triblock copolymer (e.g., Poloxamer 188, Poloxamer 407 and Poloxamer 305). In some embodiments, the solution further comprises a scavenger which can, for example, simplify viral vector manufacturing workflow by separating the product of interest from impurities during cell lysis. Examples of suitable scavengers include ethylenediaminetetraacetic acid (EDTA), ethanol, polyethyleneimine (PEI) and its derivatives and deoxythymidine triphosphate (DTTP). The PEI derivatives include acetylation and quaternization of PEIs, an addition of methyl, ethyl or propyl groups to the secondary amine group via alkyl halide, alkyl anhydride or alkyl acetyl chloride.

In some embodiment, the purified detergent composition has a purity of greater than 95%, and the common impurities that are known to have toxicity such as ethylene oxide (<5 ppm) and dioxane (<10 ppm) are controlled. The purification process includes distillation, reactive distillation, filtration, column chromatographic adsorption or combinations thereof.

In some embodiments, the membrane rupture solution may also contain other stabilizers such as sucrose, trehalose, dextrose, polyethylene glycol (PEG), sodium chloride (NaCl), and magnesium chloride (MgCl₂).

In another aspect, the invention also includes methods of producing highly pure detergents and of preparing cell rupture solutions. In another aspect, the invention also includes methods of lysing mammalian and insect cell lines, and of inactivating enveloped viruses. In another aspect, the membrane rupture solution can be useful for isolating viral vector particles.

In another aspect, membrane rupture compositions can be useful in separating full capsids (genome containing) from empty capsids and other product and process related impurities.

### BRIEF DESCRIPTION OF THE DRAWINGS

FIG. 1: is a graph showing cell lysis of HEK-293 (Panel A) and SF9 (Panel B) cells by solution A and Triton X-100. Percent cell viability was measured following 1 hour of cell lysis.

FIG. 2: is a graph showing cell lysis of HEK-293 (Panel A) and SF9 (Panel B) cells by solution A and solution B. Percent cell viability was measured following 1 hour of cell lysis.

FIG. 3: is a graph demonstrating the synergistic effect of solution A and Polysorbate 20 for cell lysis of HEK-293 (Panel A) and SF9 (Panel B) cells. Percent cell viability was measured following 1 hour of cell lysis.

FIG. 4: is a graph depicting cell lysis of HEK-293 (Panel A) and SF9 (Panel B) cells by solution B and solution C. Percent cell viability was measured following 1 hour of cell lysis.

FIG. 5: is a graph depicting the foam height of membrane rupture solutions Triton X-100, solution A, solution B and solution C. The measurement was conducted at 0.1% membrane rupture solution concentration according to ASTM D 1173, Standard Test Method for Foaming Properties of Surface-Active Agents.

FIG. 6: is a graph depicting cell lysis of HEK-293 cells by Solution C in 50 mM Tris at pH 4.0 and in the cell culture media. Percent cell viability was measured following 1 hour of cell lysis.

FIG. 7: is a graph depicting virus titer using membrane rupture solutions Triton X-100, solution A, solution B and solution C. The titer was measured using qPCR method.

### DETAILED DESCRIPTION OF THE INVENTION

The present invention relates to compositions and methods for use in extracting and isolating non-lytic virus, protein, and peptide molecules from the host cell. More specifically, the invention relates to such compositions and methods that are useful in the extraction and isolation of viral vectors from host cells (e.g., mammalian and insect cells) via lysis.

In one aspect, the invention provides membrane rupture solutions. These solutions can be used for lysing host cells and viral inactivation. The compositions comprise one or more purified, biodegradable, and environmentally friendly detergent(s) in the concentration range from about 0.001% (w/v) to about 1% (w/v). Examples of suitable detergents include alcohol ethoxylates (e.g., TDA 9, Tergitol 15-S-9), Sorbiton ethoxylate (e.g., Polysorbate 20, Polysorbate 80), and triblock copolymer (e.g., Poloxamer 188, Poloxamer 407 and Poloxamer 305).

In some embodiments, the compositions also include at least one scavenger in the concentration range of 0.01% (w/v) to about 1% (w/v). Examples of suitable scavengers include EDTA, ethanol, DTTP and PEI or its derivatives. Specifically, PEI can be a linear or branched structure with a molecular weight from 3000 KD to 100,000 KD. The derivatization of PEI may include addition of alkyl groups such as, for example, a methyl, ethyl, or propyl group at the secondary amine group using currently known acetylation or alkylation techniques utilizing reagents such as, for example, acetic anhydride or acetyl chloride.

In some embodiments, the composition may further include one or more of stabilizers. Examples of suitable stabilizers include sucrose, trehalose, dextrose, polyethylene glycol (PEG) of various chain lengths, sodium chloride (NaCl), and magnesium chloride (MgCl₂) at a concentration range of approximately 0.001M to about 2M. The chain length of PEG could vary from 100 to 10,000 monomer units.

In some other embodiments, the composition can further include a buffer in an amount sufficient to maintain pH at a range from about 3.0 to about 9.0. The composition can be an aqueous solution, or an aqueous concentrate. In one aspect, the invention provides a method for recovering viral vectors from host cells, such as mammalian and insect cells. The method comprises the steps of: (a) providing a method for cell growth thereby providing a cell preparation; (b) providing a cell rupture solution comprising one or more detergent(s) in the concentration range of about 0.01% to about 1% (w/v), wherein at least one of the detergents has a surface activity property that is vital for viral vector/protein stabilization against shear stress (optionally, comprising scavengers and stabilizers); (c) contacting the cell preparation with the cell rupture solution, wherein cell lysis occurs; (d) conducting a cell viability analysis; (e) conducting a DNA measurement; and (f) conducting virus titer.

In one embodiment, the in-use cell rupture solution comprises about 0.25% (w/v) TDA9 (solution A) in an aqueous solution where solution pH is about 3.0 to 9.0. TDA9 is a biodegradable nonionic detergent derived from isotridecyl alcohol and ethoxylated to an average of nine moles of ethylene oxide. It shows excellent rapid wetting properties, relatively low foaming levels, good detergency and versatility as an emulsifier, dispersant, and solubilizer. The cell rupture solution was tested both for HEK 293 and SF9 cell lines. The cell concentration was >5×10⁶ cells/ml. A cell harvest in cell culture media was spiked with the concentrated stock of cell rupture solution to reach a final cell rupture solution concentration ranging from 0% to 0.5% and was incubated for about 5 minutes to about 1 hour at 15° C. to 30° C. The concentration of the stock solution could be 1-500-fold higher than the target concentration. After the incubation period, cell viability of cells was analyzed by a Vi-Cell reader. Cell viability analysis demonstrated complete cell lysis in about 5 min. Incubation beyond 5 minutes did not provide any benefit nor did it cause any harm.

In one aspect, the present invention provides a biodegradable environmentally friendly detergent that can lyse both mammalian and insect cells similarly to the currently used detergent Triton X-100. Since Triton X-100 will not be available for use beginning January 2021, the current invention is important for viral-vector and other non-lytic cellular products. In this invention, the detergent is highly pure and free from product-related impurities such as polyethylene glycol and free ethylene oxide, which may have an adverse impact on the product of interest. Detergents used here are low in both microbial contaminants and ethylene oxide.

Reduction of microbial contamination during the purification step of TDA9 reduces the burden on downstream processing to remove impurities such as polyethylene glycol and free ethylene oxide during purification steps such as chromatography. Removal of ethylene oxide from TDA9 is critical as ethylene oxide is a hazardous substance [8], which is carcinogenic and requires extra precaution during handling. Additionally, TDA9 is an environmentally compatible detergent which meets OECD guideline 301F for readily biodegradable material. Short term (acute) toxicity of the detergent is classified as category 2, which does not require a warning label, and LC₅₀ and EC₅₀ values are higher than 1.00 and less than 100. Toxicity to aquatic environments is tested in all three trophic levels, according to OECD 203 (toxicity to fish), OECD 202 (toxicity to aquatic invertebrates), and OECD 201 (toxicity to algae).

In another aspect of this embodiment of the invention, the cell rupture solution is comprised of about 0.05% TDA9 and about 0.20% (w/v) polysorbate 20 (solution B) in an aqueous solution. Polysorbate 20, also known as Tween 20, is a sorbitan ethoxylate-type nonionic surfactant formed by the ethoxylation of sorbitan ester before the addition of lauric acid. It is relatively non-toxic and used in several domestic, scientific, and pharmacological applications. As the name implies, the ethoxylation process leaves the molecule with 20 repeat units of polyethylene glycol. The solution pH is about 3.0 to about 9.0. This cell rupture solution was tested both for HEK 293 and SF9 cell lines. The cell concentration was >5×10⁶ cells/mL. A cell harvest in cell culture media was spiked with the concentrated stock of cell rupture solution to reach a final cell rupture solution concentration ranging from 0% to 0.5% and was incubated for a period of about 5 minutes to about 1 hour at 15° C. to 30° C. After the incubation period, cell viability was analyzed by a Vi-Cell reader. Cell viability analysis demonstrated complete cell rupture in about 5 min. Solution B demonstrated comparable lysis to Solution A for the lysis solution concentration of 0.1% (w/v) or higher. As illustrated in example 3, the rupture solution B unexpectedly showed higher cell lysis than theoretically expected utilizing mathematical calculation based on component concentration. This synergetic performance phenomenon driving better cell lysis. The membrane rupture solutions of the instant invention have a low TDA9 concentration vis-&-vis Polysorbate 20. In one embodiment, the Polysorbate 20 used in the instant invention is super refined low impurity polysorbate 20 from Avantor J. T. Baker brand. In another embodiment, solution B has lower foam height compared to solution A or Triton X-100, offering operational flexibility.

In another embodiment of the invention, the membrane rupture solution comprises about 0.05% TDA9, about 0.20% (w/v) polysorbate 20 and about 0.005% (w/v) poloxamer 188 (solution C). Poloxamers are non-ionic tri-block copolymers composed of a central hydrophobic chain of polyoxypropylene (poly (propylene oxide)) flanked by two hydrophilic chains of polyoxyethylene (poly (ethylene oxide)). Poloxamer 188 protects the product against shear stress, for example, protects the viral vector during lysis and purification. The degradation of viral vector due to shear induced stress has been reported in the literature [9]. Poloxamer 188 impacts cell lysis as it has been reported to be a cell membrane stabilizer and also has an application as a membrane resealing reagent in biomedical engineering [10,11]. The effect of poloxamer 188 on the ability of the membrane rupture solution to lyse cells was evaluated. In this invention, the solution pH was about 3.0 to about 9.0. Surprisingly, the inventors of the instant invention observed that poloxamer 188 does not adversely impact cell lysis when it is used as part of membrane rupture solution. The cell rupture solution was tested both for HEK 293 and SF9 cell lines. The cell concentration was >5×10⁶ cells/mL. A cell harvest in cell culture media was spiked with the concentrated stock of cell rupture solution to reach a final cell rupture buffer concentration ranging 0% to 0.5% and was incubated for a period of about 5 minutes to about 1 hour at 15° C. to 30° C. After the incubation period, cell viability of cells was analyzed by a Vi-Cell reader. Cell viability analysis demonstrated complete cell rupture in about 5 min. Incubation beyond 5 minutes did not provide any benefit nor did it cause any adverse impact. Results demonstrated that cell lysis was comparable with solution B and solution C. In one embodiment, the invention provides a lysis buffer that not only lyses cells completely but also protect the product of interest in the lysate from shear induced damage that it might experience during the manufacturing process. Poloxamer 188 used in this study also has a long history of use in the pharmaceutical industry and is listed as a GRAS excipient [1,12,13]. Poloxamer 188 used in this invention may be from Avantor J. T. Baker brand or could be sourced from other vendors.

In another embodiment of the invention, the membrane rupture solution comprises 0.05% TDA9, 0.20% (w/v) polysorbate 20, 0.005% (w/v) poloxamer 188 and 0.1% PEI (solution D). PEI, polyethylenimine, is a polymer with a repeating unit composed of an amine group and two carbon aliphatic CH₂CH₂ spacers. Although PEI has been used to selectively precipitate DNA [14] it is not used in cell lysis solutions due to interference of its activity with cell membrane constituents such as phospholipids and membrane protein. In this invention, the inventors have surprisingly discovered that that the presence of PEI in the lysis buffer significantly reduced DNA in the lysate following cell lysis without interference from cell membrane constituents and removal of cell debris with no significant change in cell lysis intensity. The removal of DNA by PEI was calculated by comparing the total DNA in the lysate, lysed by solution D, to PEI-free lysis solutions. DNA in the lysate was measured by Denovix Fluorescence Quantification Assay (DeNovix dsDNA Broad Range Kit). PEI can significantly improve viral vector yield, and in turn, simplify manufacturing workflow. PEI can selectively precipitate product related impurities (e.g., empty capsids and DNA), while retaining full viral vector capsids in the lysate.

In another embodiment of the invention, viral vector producing cells were lysed with solution A, solution B and solution C. Briefly, cells and lysis buffer mixture were exposed to shear stress and then purified. The yield was better for the cells that were lysed with solution C than solution A and solution B, as measured by qPCR. The higher titer for poloxamer-containing solution demonstrates viral vector protection by poloxamer 188.

In another embodiment of the invention, lysis of cells by solution D surprisingly precipitated both DNA impurities and AAV virus. Since the PIs of empty versus full capsids, as well as DNA impurities, are different, the lysis solution conditions can be optimized to selectively precipitate empty viral capsids and DNA impurities from the full capsid, a virus of interest in the solution while lysis.

This invention has significant impact on the manufacturing of non-lytic cell products. In some embodiments, the compositions comprise poloxamer 188 to minimize product damage due to shear stress during downstream processing and comprise PEI to selectively reduce empty capsids and DNA impurities from the lysate. This significantly improves the current viral vector manufacturing workflow, resulting in overall yield improvement and lower cost of treatment.

A concentrated stock of the membrane rupture solution can be provided for use and diluted to the final concentration in the working solution. The stock of the concentrated solution may vary from about 1-500-fold, or about 5-200, of the use concentration.

In one example, the in-use cell rupture solution comprises 0.25% (w/v) ethoxylated alcohol (solution A). In another example, the in-use membrane rupture solution contains 0.05% ethoxylated alcohol and 0.20% (w/v) polysorbate 20 (solution B) in an aqueous solution. In another example, the in-use membrane rupture solution contains 0.05% ethoxylated alcohol, 0.20% (w/v) polysorbate 20 and 0.005% (w/v) poloxamer 188 (solution C). In another example, the in-use membrane rupture solution contains 0.05% ethoxylated alcohol, 0.20% (w/v) polysorbate 20 and 0.005% (w/v) poloxamer 188 and 0.10% PEI (solution D). The solution pH is approximately 3.0 to about 9.0. The conductivity of the solution is approximately 5 to about 200 mS/cm. In some embodiments, conductivity is adjusted using salt solutions such as sodium chloride or magnesium chloride in the range of about 5 to 200 mS/cm.

The inventors have also unexpectedly observed that certain compositions of membrane rupture solution provide lower foam height, thereby providing ease of use and improving viral particle recovery.

TABLE 1
Elucidated composition of in-use membrane
rupture solution in cell suspension

| | Composition and final concentration |
|---|---|
| Membrane rupture solution | of membrane rupture solution |
| Rupture solution A | 0.25% TDA9 |
| Rupture solution B | 0.05% TDA9 |
| | 0.20 Polysorbate 20 |
| Rupture solution C | 0.05% TDA9 |
| | 0.20 Polysorbate 20 |
| | 0.005% Poloxamer 188 |
| Rupture solution D | 0.05% TDA9 |
| | 0.20 Polysorbate 20 |
| | 0.005% Poloxamer 188 |
| | 0.1% PEI |

The method of use of the membrane rupture solution includes the addition of the stock lysis solution to the cell suspension either in cell culture media or in a suitable buffer system. The final membrane rupture solution concentration in cell suspension is greater than about 0.01% (w/v), preferably about 0.25% (w/v), and in some embodiments, greater than about 1% (w/v). The stock membrane rupture solution can be directly added to cells in culture media, or the harvested cells may be resuspended in a suitable buffer solution at the target final concentration. The pH of the solution may vary from about 3.0 to about 9.0. The conductivity of solution may vary from about 5-200 mS/cm. The mixture of cell suspension and membrane rupture solution should be held for a minimum of about 5 min, and preferably for about 1 hour for cell lysis to be complete.

A typical example of cell lysis is accomplished during the manufacturing process of non-enveloped AAV viral vectors. A typical downstream process first includes centrifugation and cell lysis, where cells are concentrated into a slurry by centrifugation and then lysed to release viruses by detergent, freeze/thaw, or mechanical homogenization before further purification to remove product and process-related impurities. Other unit operations in viral vector downstream processing include nucleic acid removal, where lysates generated following cell lysis are digested with an endonuclease to reduce nucleic acid contaminants; solid removal by centrifugation or microfiltration to remove cell fragments and debris prior to chromatographic purification; affinity chromatography to remove of HCPs and any serum protein impurities; removal of genome-containing infectious AAV viruses from empty, non-infectious capsids, either by cesium chloride gradient ultracentrifugation procedures or ion-exchange chromatography; and final polishing to further reduce HCPs or other low molecular weight contaminants using core-bead adsorbents [15].

A typical example of viral inactivation is removal of virus from blood plasma [16,17]. Health authorities mandate removal of virus from blood plasma prior to patient administration. This is a critical safety step to minimize the likelihood of transfusion of transmissible infectious agents (such as hepatitis) via blood components. Another example of viral inactivation of cellular derived products occurs during production of recombinant products such as monoclonal antibodies. Monoclonal antibody manufacturing includes several unit operations to inactivate/remove viruses from the product. Demonstration of viral clearance is mandatory for recombinant products according to FDA and other regulatory agency guidelines [18]. The viral inactivation process by detergent involves the addition of detergent solution to the cellular derived product at or above CMC and hold for several hours. However, inactivated virus must be removed from the product before human use by filtration, chromatography and/or any other method [16,17].

While there have been described what are presently believed to be the preferred embodiments of the present invention, those skilled in the art will realize that other and further changes and modifications may be made without departing from the spirit of the invention, and it is intended to claim all such modifications and changes as come within the true scope of the invention. The present invention is illustrated in further details by the following non-limiting examples.

### Examples

The materials and methods used in this disclosure are the following:

### TDA9 Purification

TDA9 purification was performed to reduce product-related impurities, specifically ethylene oxide. The purification process was comprised of (1) heating raw material to 50-180° C. while mixing with nitrogen or any inert gas or applying vacuum, (2) cooling down the product to 15-40° C. while mixing with inert gas, (3) addition of 10-50 volume percent of water to create a completely homologized solution, (4) heating of the solution to 40-120° C. to complete hydrolysis of remaining volatile impurities, and (5) filtration of mixture with 0.2-1.0 μm filter.

### Cell Growth:

HEK 293 cells for the study were grown in Expi293 expression medium (Gibco A14351-01) in a vented non-baffled shake flask. The incubator was set to 37° C. The shaker speed was 125 rpm. The CO₂ was maintained at 6% and the relative humidity was >60%. The target cell count of >5×10⁶ viable cells/mL was achieved in 4-5 days. SF9 cells for the study were grown in SF-900 II SFM (Invitrogen/Gibco 10902) media in a non-vented non-baffled shake flask. The incubator was set at 27° C. and 125 rpm. The target cell count of >5×10⁶ viable cells/mL was achieved in 4-5 days.

### Sample Preparation for Cell Lysis:

For the cell lysis study, 2 mL of cells were added to each of 10×15 mL tubes under a sterile biosafety cabinet. Each tube was spiked with 0% (control, no detergent), 0.0025%, 0.005%, 0.0075%, 0.01%, 0.02%, 0.05%, 0.1%, 0.25%, and 0.5% of cell rupture solution using a concentrated stock. Cell counts and percent cell viability were determined at 15 min, 30 min, and 1 hour time points for each sample using the ViCell cell viability analyzer.

### Cell Viability Study:

Cell viability was assessed by a Vi-Cell XR using trypan blue exclusion staining. The Vi-Cell XR Cell Viability Analyzer is a video imaging system used to analyze cellular viability. It automates the trypan blue exclusion protocol, in which dead cells take up the dye whilst live cells do not [19]. The sample is delivered to a flow cell and camera for imaging, where differences in the grey scale between live and dead cells are determined by the software. For every sample, the instrument requires 500 μL of sample volume, which is mixed 1:1 with trypan blue and takes up to 50 images to determine the cell concentration and viability.

### Foam Height Measurement:

The foam height determination of various membrane rupture solutions was carried out according to ASTM D 1173, Standard Test Method for Foaming Properties of Surface-Active Agents[20]. All foam height measurements were carried out at 25° C. (room temperature) using a Ross-Miles Foam Apparatus (VWR Cat #14007-876) by Wilmad-Labglass.

### DNA Measurement:

DNA was measured using dsDNA broad range fluorescence assay kit by DeNovix. The kit enables detection of dsDNA samples with a standard detection range from 2 to 2000 ng total mass in 200 μL volume. The spectral properties of the dye are excitation/emission of 350/460 nm in the presence of dsDNA. It is a broad range 2-point assay. DNase-free pipette tips were used for this assay (VWR Cat #s 89174-528, 89174-530, 89714-524); all reagents were equilibrated to room temperature. Working solution was prepared in a 2 mL VWR micro tube with cap (sterile, DNase-free, Cat #16466-042): 1 mL assay buffer+10 μL dye+10 μL enhancer. All standards (0 ng and 200 ng) and samples were prepared by adding 190 μL working solution to 10 μL standard or 10 μL of sample in PCR tubes. Assay tubes were incubated at room temperature for 5 minutes. Preconfigured standards were selected on the menu and then “generate new standard curve” was chosen. The 0 ng/μL and 200 ng/μL standards were then read. Samples were measured following the generation of this curve. Relative fluorescence units and DNA concentration were recorded for both samples and standards.

### Example 1: Cell Lysis by Membrane Rupture Solution A

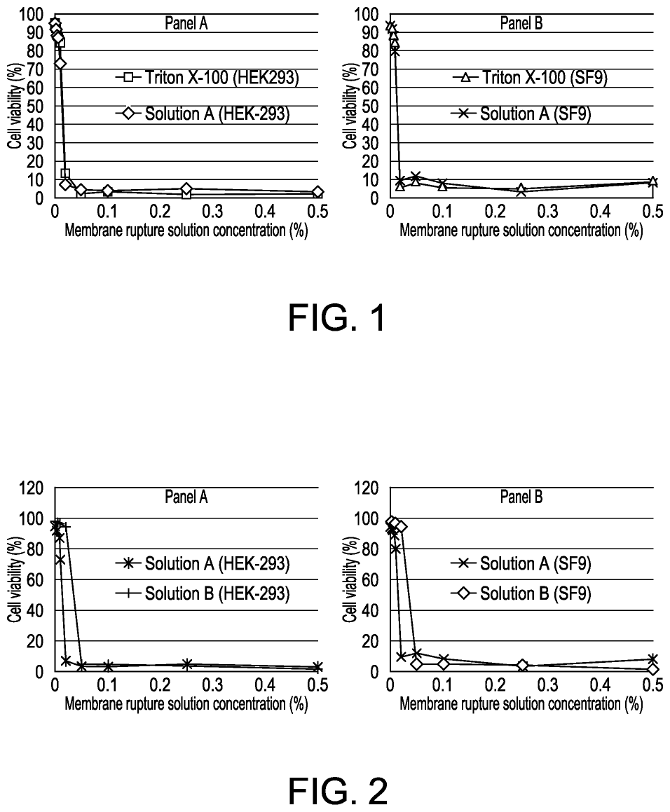

Cell lysis of HEK 293 and SF9 cells by membrane rupture solution A and Triton X-100 was performed. Cell counts prior to lysis were measured for both cell types. Samples were prepared and analyzed as discussed earlier. The control experiment with Triton X-100 was conducted identically to that of solution A. Cell counts and percent cell viability were determined following 1 hour of incubation using the ViCell cell viability analyzer. Results of cell lysis are shown in FIG. 1. For both cells lines, cell lysis was complete at the detergent concentration of 0.05% (w/v) and the results were comparable for solution A and Triton X-100. Detergent concentration beyond 0.05% did not provide any advantage.

### Example 2: Cell Lysis by Membrane Rupture Solution B

Cell lysis of HEK 293 and SF9 cells by solution A and solution B (solution A+PS20) was performed. Cell counts prior to lysis were measured for both cell types. Samples were prepared and analyzed as discussed earlier. The results of cell lysis for 1 hour incubated samples are shown in FIG. 2. Complete cell lysis was achieved for the solution B at a concentration of 0.1%. Concentrations beyond 0.1% did not provide any advantage. Although cell lysis was achieved by solution A at a lower concentration than solution B, the small difference has practically no value since cell lysis is usually performed at a concentration above 0.1% [5,6,21,22]. Therefore, at the typical concentration of use, solution A and the solution B are comparable in performance. One of the advantages of using solution B over solution A is that it generates less foam height compared to solution A (Example 5).

### Example 3: Synergistic Effect of Solution a and Polysorbate 20

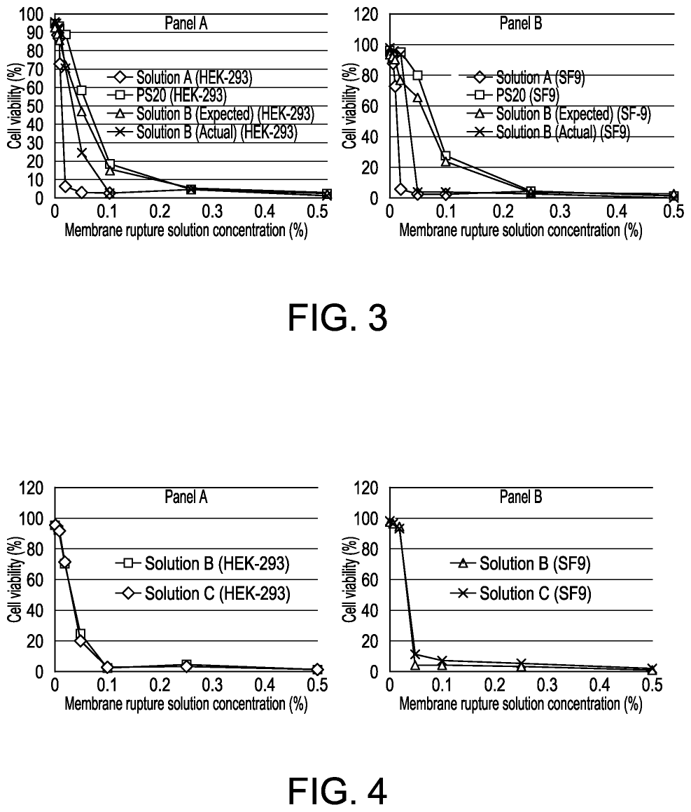

Cell lysis of HEK 293 and SF9 cells by solution A, PS20 and solution B (solution A+PS20) was performed. Cell counts prior to lysis were measured for both cell types. For the sample preparation, three stocks of membrane rupture solution (solution A, PS20 and solution B) were used. Samples were prepared and analyzed as discussed earlier. Results are shown in FIG. 3.

This study was conducted to evaluate the synergic effect of Solution A and PS20. To examine this, experimental cell lysis by Solution B was compared with the expected cell lysis. The expected response of solution B was calculated as follows:

Expected cell lysis (%) by solution B=0.2*lysis by solution A+0.8*lysis by PS20.

As shown in the figure, experimentally observed lysis by solution B is significantly better than the expected lysis, suggesting a synergistic effect between solution A and PS20.

### Example 4: Cell Lysis by Membrane Rupture Solution C

Cell lysis of HEK 293 and SF9 cells by solution B and solution C (solution B+Poloxamer) was performed. Cell counts prior to lysis were measured for both cell types. Results of cell lysis for 1 hour incubated samples are shown in FIG. 4. Cell lysis was comparable for both solution B and solution C, demonstrating that poloxamer is compatible with solution B solution and does not cause any adverse impact on cell lysis. This is a very important invention as poloxamer is expected to provide protection to the product of interest, in this case viral vectors, during harsh manufacturing conditions, without compromising cell lysis.

### Example 5: Foam Height Comparison of Membrane Rupture Solutions

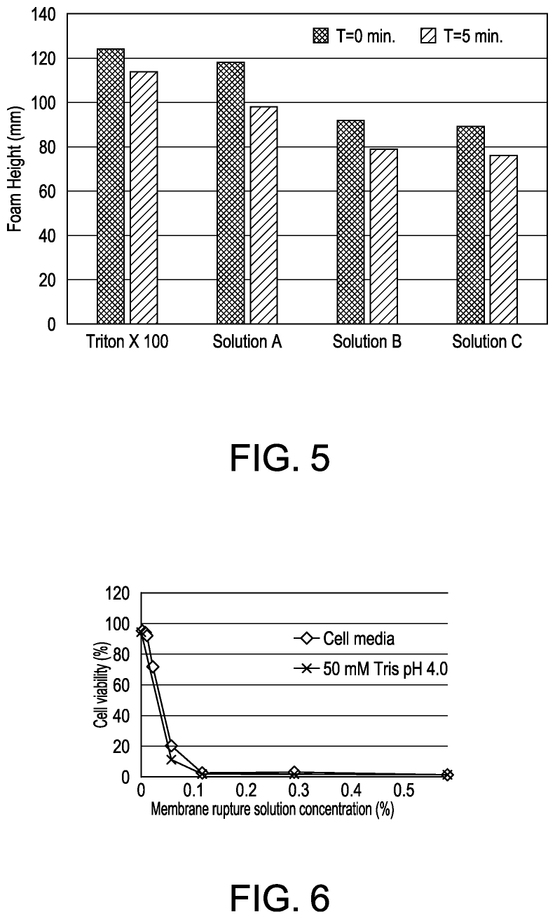

Minimizing foam height is a common challenge when using detergents. Ideally, the cell lysis solution should be able to lyse cells without creating too much foam. In this experiment, the foam height of Triton X-100, solution A, solution B and solution C was compared. Foam height determination was performed using a Ross-Miles Foam Apparatus (VWR Cat #14007-876) by Wilmad-Labglass. FIG. 5 shows the foam height at time zero and after 5 minutes of hold time. Foam height of solution D was not measured because PEI is not a detergent, and therefore, is not expected to impact the foam height of solution C. The foam height of cell lysis solutions B and C is significantly lower than the foam height of solution A and Triton X-100.

Since solution A, solution B and solution C has demonstrated comparable lysis capability to Triton X-100 and solution B and solution C have lower foam height than both Triton X-100 and solution A, the membrane rupture solutions solution B, solution C and solution D are better alternatives to Triton X-100 than solution A.

### Example 6: Removal of DNA During Cell Lysis

HEK 293 and SF9 cell lysis was performed by both freeze/thaw method and by rupture solution method, with addition of solution B, solution C and solution D. The DNA concentration in HEK cells prior to and following lysis was measured. Cell lysis by freeze/thaw was done by freezing the cell suspension at −80° C. for 30 minutes and thawing it in a 37° C. water bath for 15 minutes. Freeze-thaw was repeated for 4 cycles. The control and freeze-thaw samples were spun for 10 minutes at 500×g to remove the cell debris. The DNA of supernatant was measured by a dsDNA broad range fluorescence assay kit. Results are shown in Table 2. The baseline was corrected using cell culture media. For cell lysis via the rupture solutions, solution B, solution C, solution D were spiked into each of 2 mL cell suspensions. A period of 15 minutes was allowed for cell lysis to occur prior to DNA quantification measurements. Results are shown in Table 2. The total DNA concentration was highest in the lysate where cell lysis was performed by the freeze/thaw method. The DNA concentration in solution B and solution C lysates was slightly lower than the DNA concentration in the freeze/thaw lysate. The DNA concentration in solution D was nearly zero. This is an important invention as it will reduce the burden on downstream processing.

TABLE 2
DNA content in cell lysate following cell lysis by various methods

| | | HEK cells | SF9 cells |
|---|---|---|---|
| | | DNA | DNA |
| Cell lysis | | Concentration | Concentration |
| method | Composition | (ng/μL) | (ng/μL) |
| Control | N/A | 2 | 4 |
| Freeze-thaw | N/A | 18 | 9 |
| Solution B | 0.02% TDA9 + 0.08% | 13 | 15 |
| | Polysorbate 20 | | |
| Solution C | 0.02% TDA9 + 0.08% | 13 | 15 |
| | Polysorbate 20 + 0.005% | | |
| | Poloxamer | | |
| Solution D | 0.02% TDA9 + 0.08% | Not detected | 7 |
| | Polysorbate 20 + 0.005% | | |
| | Poloxamer + 0.1% PEI | | |

### Example 7: Cell Concentration Dependent DNA Removal

HEK 293 cell lysis was performed at cell concentrations of 5×10⁶ cells/mL, 10×10⁶ cells/mL, 20×10⁶ cells/mL and 40×10⁶ cells/mL in 50 mM Tris, pH 6.5 buffer using solution B, solution C, solution D lysis buffer as per method as described in Example 6. Results are shown in Table 3. The DNA concentration in cells lysed by solution D was significantly lower than the cells lysed by solutions B and C, demonstrating that the PEI containing lysis buffer can effectively remove DNA from the cell suspension. As expected, the DNA concentration increased with cell concentration after lysis with solution B and solution C; however, the DNA concentration was comparable for lysis with solution D, suggesting that DNA removal by PEI in the lysis buffer mixture of solution D is independent of cell concentration in the range of 5×10⁶-40×10⁶ cells/mL.

TABLE 3
Cell concentration dependent DNA removal by PEI

| Cell | | DNA concentration (ng/μL) | | | |
|---|---|---|---|---|---|
| lysis | | 5X10⁶ | 10X10⁶ | 20X10⁶ | 40X10⁶ |
| method | Composition | cells/mL | cells/mL | cells/mL | cells/mL |
| Control | N/A | 1 | 1 | 2 | 5 |
| Solution B | 0.05% | 17 | 13 | 19 | 25 |
| | TDA9 + | | | | |
| | 0.2% | | | | |
| | Polysorbate | | | | |
| | 20 | | | | |
| Solution C | 0.5% | 18 | 17 | 20 | 27 |
| | TDA9 + | | | | |
| | 0.2% | | | | |
| | Polysorbate | | | | |
| | 20 + 0.005% | | | | |
| | Poloxamer | | | | |
| Solution D | 0.05% | 3 | 4 | 2 | Not |
| | TDA9 + | | | | detected |
| | 0.2% | | | | |
| | Polysorbate | | | | |
| | 20 + 0.005% | | | | |
| | Poloxamer + | | | | |
| | 0.1% PEI | | | | |

### Example 8: Compatibility of Lysis Buffer with Sucrose and Magnesium Chloride

Sucrose and magnesium chloride are widely used in viral vector manufacturing to improve overall yield and product quality [23]. The study was conducted to demonstrate that the disclosed cell membrane rupture solution is compatible with sucrose and magnesium chloride (MgCl₂) and does not have any adverse impact on cell lysis. The cell stock was divided in two tubes and spun for 10 minutes at 100×g. The supernatant was removed from the first tube and the solid pellet was resuspended in 50 mM Tris, pH 6.5. The supernatant was removed from the second tube and the pellet was resuspended in 50 mM Tris, pH 6.5, 2 mM MgCl₂ and 1 M sucrose. The cell concentration of the suspension was measured to be approximately 5×10⁶ cells/ml, as measured by ViCell. For cell lysis, solution C was spiked into 2 mL of cells using a concentrated stock to obtain final detergent concentrations of 0%, 0.05%, 0.1%, 0.25% and 0.5%. Cell counts and percent cell viability were determined after 1 hour incubation time for each sample using the ViCell cell viability analyzer. Cell viability results as a function of detergent concentration are shown in Table 4. Cell viability without detergent was lower for cells suspended in buffer containing MgCl₂ and sucrose than buffer alone, suggesting that MgCl₂ and sucrose can partially lyse cells without any detergent. This is not an unexpected result, due to the potential of osmotic shock leading the cells to rupture. However, partial (45%) lysis is not sufficient to replace detergent use. The osmolality values of 50 mM Tris, pH 6.5 and 50 mM Tris, pH 6.5, 2 mM MgCl₂ and 1M sucrose are 114 mOsm/Kg and 1462 mOsm/Kg, respectively. At lower detergent concentrations, the membrane rupture solution is slightly more effective in the MgCl₂ and sucrose free buffer than in the presence of MgCl₂ and sucrose. However, at the typical concentration of detergent use, the percent lysis is comparable in both cases, suggesting that MgCl₂ and sucrose, which are used as stabilizers, do not have any adverse impact on cell lysis. This is a significant observation because the use of sucrose and MgCl₂ during cell lysis is expected to stabilize viral vector and improve recovery.

TABLE 4
Effect of magnesium chloride and sucrose
on cell lysis by solution C

| Membrane rupture | Cell viability (%) | |
|---|---|---|
| solution concentration | Without | With |
| (%) | MgCl₂ and sucrose | MgCl₂ and sucrose |
| 0 | 92.5 | 56.3 |
| 0.05 | 9.0 | 14.7 |
| 0.1 | 5.3 | 1.6 |
| 0.25 | 3.5 | 0.5 |
| 0.5 | 2.0 | 0.6 |

### Example 9: pH Dependent Cell Lysis by Membrane Rupture Solution

Cell lysis of HEK 293 by solution C was performed at pH 4.0 in a 50 mM Tris buffer and compared with cell lysis in cell media (pH 7.2-7.4) as discussed in Example 4. The appropriate volume of required cells in cell culture media was spun for 10 minutes at 100×g. The supernatant was removed, and the solid pellet was resuspended in the appropriate volume of 50 mM Tris, pH 4.0. Cell concentration of the suspension was measured to be approximately 5'10⁶ cells/mL. A concentrated stock of solution C was used to spike the cell suspension. 2 mL of cells were added to each of 5×15 mL tubes under a sterile biosafety cabinet. Each tube was spiked with 0% (control, no detergent), 0.05%, 0.1%, 0.25%, and 0.5% final concentration of detergent from the detergent stock solution. Cell counts and percent cell viability were determined after 1 hour incubation time for each sample using the ViCell cell viability analyzer. Results of cell lysis for 1 hour incubated samples are shown in FIG. 6. Cell lysis at pH 4.0 was comparable to cell lysis in the cell culture media, demonstrating that the cell lysis low pH does not have any adverse impact on the cell lysis by membrane rupture solution.

### Example 10: pH Dependent DNA Removal by Membrane Rupture Solution

DNA removal by membrane rupture solutions was evaluated at pH 4.0 in a 50 mM Tris buffer in HEK 293 cells and compared to DNA removal in cell culture media (pH 7.2-7.4) as discussed in Example 8. The appropriate volume of required cells in cell culture media was spun for 10 minutes at 100×g. The supernatant was removed, and the solid pellet was resuspended in an appropriate volume of 50 mM Tris, pH 4.0. Cell concentration of the suspension was measured to be approximately 5×10⁶ cells/mL. Cell lysis was performed with addition of solution B, solution C and solution D to make a final membrane rupture solution concentration of 0.25%. The DNA concentration in solution prior to lysis was measured as a control. The DNA content of the supernatant was measured by a dsDNA broad range fluorescence assay kit. Results are shown in Table 5. The baseline was corrected using 50 mM Tris pH 4.0 buffer. A period of 15 minutes was allowed for cell lysis to occur prior to DNA quantification measurements. The total DNA concentration was higher in the lysate where cell lysis was performed by either solution B, or solution C, than solution D. The DNA in the lysate was comparable at pH 4.0 to the DNA in the lysate in the cell media as shown in example 8. Cell density was comparable in both examples (˜5×10⁶ cells/mL). This result demonstrates that the DNA generated during lysis and its subsequent removal by membrane rupture solutions at pH 4.0 is comparable to DNA generated during lysis and its subsequent removal by membrane rupture solutions in cell culture media (pH 7.2-7.4), demonstrating that low pH does not have any adverse impact on DNA removal by the membrane rupture solution comprised of solution D.

TABLE 5
Effect of pH on DNA removal by membrane rupture solutions

| | | Cell media, pH | |
|---|---|---|---|
| | | (7.2-7.4) | pH 4.0 |
| | | DNA | DNA |
| Cell lysis | | concentration | concentration |
| method | Composition | (ng/μL) | (ng/μL) |
| Control | N/A | 1 | 0 |
| Solution B | 0.05% TDA9 + 0.2% | 17 | 10 |
| | Polysorbate 20 | | |
| Solution C | 0.05% TDA9 + 0.2% | 18 | 14 |
| | Polysorbate 20 + 0.005% | | |
| | Poloxamer | | |
| Solution D | 0.05% TDA9 + 0.2% | 3 | 0 |
| | Polysorbate 20 + 0.005% | | |
| | Poloxamer + 0.1% PEI | | |

### Example 11: Comparison of Viral Vector Titers of Membrane Rupture Solutions

Transfection of adherent HEK293T cells was carried out using PEI max mixed 4:1 with plasmid DNA in the ratio of 2:1.6:1, for helper, R2C2 plasmids, and GFP plasmids, respectively. On day 5 post transfection, lysis experiments were carried out by spiking concentrated lysis solutions into the cell suspensions. For lysis of cells transfected with rAAV2, 2 mL of cell suspension with a concentration of 2 million cells per mL was added to 5 ml centrifuge tubes and 20 μL of 100× concentrated Triton X-100, solution A, solution B, and solution C was added to reach a final detergent concentration of about 0.25%. All four mixtures were then exposed to shear stress to mimic stress conditions that could be experienced during a typical manufacturing process such as mixing following lysis buffer and endonuclease addition. A small 12 mm size magnetic bar was inserted in each tube and then each tube was placed on a shaker. The mixtures were shaken for 60 min., 250 rpm at room temperature. Post shear stress, viral vector was harvested by centrifuging the suspensions at 14,000×g for 15 minutes at 4° C. and collecting the supernatant. A concentrated PEG 8000/NaCl mixture was added to the supernatant to reach a final concentration of 20% to flocculate the rAAV2 and the resulting suspension was incubated overnight at 4° C. Following centrifugation at 2500×g for one hour, the pellet was collected and resuspended in TAE buffer. Following chloroform and PEG8000 extractions steps, titer was determined by treating 2 μl of sample with DNAse I at 37° C. followed by lysis of the virus with a 0.5 M NaOH/0.2 M EDTA solution. Subsequently, the solution was neutralized by adding 0.5 M tris and a Promega Taq polymerase qPCR kit with primers targeting the GFP plasmid was used to quantify titer.

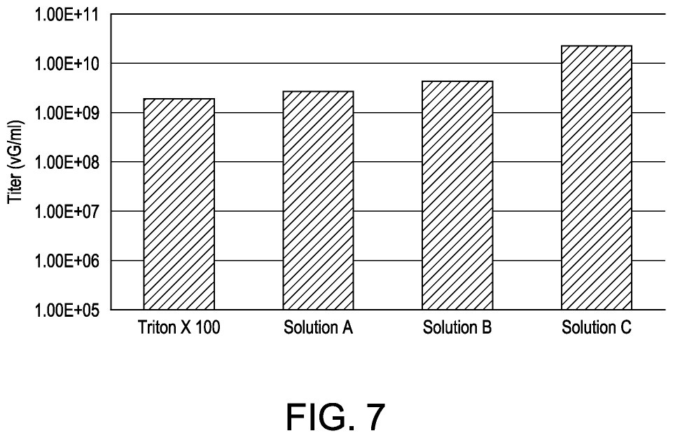

FIG. 7 shows the viral titer for different membrane rupture solutions. The chart demonstrates that titer is comparable for Triton X-100, solution A, and solution B. The titer was considerably higher for solution C, suggesting that poloxamer was able to prevent viral vector damage (titer loss) due to shear stress during mixing. This is an extremely important observation; it will allow the vector manufacturer to improve overall viral vector yield.

### Example 12: Purification of Viral Vector Using Lysis Buffer Solution D

Transfection of adherent HEK293T cells was carried out using PEI max mixed 4:1 with plasmid DNA in the ratio of 2:1.6:1, for helper, R2C2 plasmids, and GFP plasmids, respectively. On day 5 post transfection, lysis experiments were carried out by spiking concentrated lysis solution of solution D into the cell suspensions. For lysis of cells transfected with rAAV2, 2 mL of cell suspension with a concentration of 2 million cells per mL was added to 5 ml centrifuge tubes and 20 μL of 100× concentrated solution D was added to reach a final concentration of 0.25% detergent. rAAV2 was purified and titer was measured as previously described. The results demonstrated negligible recovery of viral vector in the lysate, suggesting that PEI has precipitated viral vectors along with other negatively changed impurities. This is an unexpected but extremely important observation as this may be utilized to selectively recover the viral vector of interest under appropriate solution conditions.

### REFERENCES

1. Singh S R, Zhang J, O'Dell C, Hsieh M C, Goldstein J, Liu J, et al. Effect of polysorbate 80 quality on photostability of a monoclonal antibody. AAPS PharmSciTech. 2012; 13:422-30.
2. Islam M S, Aryasomayajula A, Selvaganapathy P R. A review on macroscale and microscale cell lysis methods. Micromachines. 2017; 8.
3. He D-Y. Lysis, extraction and purification of adeno-associated virus and adenovirus from host cells. U.S. patent US010294452B2;
4. Evans R K, Nawrocki D K, Isopi L A, Williams D M, Casimiro D R, Chin S, et al. Development of stable liquid formulations for adenovirus-based vaccines. J Pharm Sci. 2004; 93:2458-75.
5. Dias Florencio G, Precigout G, Beley C, Buclez P O, Garcia L, Benchaouir R. Simple downstream process based on detergent treatment improves yield and in vivo transduction efficacy of adeno-associated virus vectors. Mol Ther—Methods Clin Dev. American Society of Gene & Cell Therapy; 2015; 2:1-7.
6. Moleirinho M G, Rosa S, Carrondo M J T, Silva R J S, Hagner-McWhirter Å, Ahlén G, et al. Clinical-grade oncolytic adenovirus purification using polysorbate 20 as an alternative for cell lysis. Curr Gene Ther. 2018; 18:366-74.
7. Farcet J, Kindermann J, Karbiener M, Kreil T R. Development of a Triton X E 100 replacement for effective virus inactivation in biotechnology processes. Eng Reports. 2019; 1:1-10.
8. Jinot J, Fritz J M, Vulimiri S V., Keshava N. Carcinogenicity of ethylene oxide: key findings and scientific issues. Toxicol Mech Methods. Informa Healthcare USA, Inc; 2018; 28:386-96.
9. Bennicelli J, Wright J F, Komaromy A, Jacobs J B, Hauck B, Zelenaia O, et al. Reversal of blindness in animal models of leber congenital amaurosis using optimized AAV2-mediated gene transfer. Mol Ther. 2008; 16:458-65.
10. G. Moloughney J, Weisleder N. Poloxamer 188 (P188) as a Membrane Resealing Reagent in Biomedical Applications. Recent Pat Biotechnol. 2013; 6:200-11.
11. Kwiatkowski, T. A.; Rose, A. L.; Jung, R.; Capati, A., Hallak, D., Yan, R.; Weisleder N. Poloxamer mediated plasma membrane repair. 2019;
12. Agarkhed M, O'Dell C, Hsieh M C, Zhang J, Goldstein J, Srivastava A. Effect of surfactants on mechanical, thermal, and photostability of a monoclonal antibody. AAPS PharmSciTech. AAPS PharmSciTech; 2018; 19:79-92.
13. Agarkhed M, O'Dell C, Hsieh M C, Zhang J, Goldstein J, Srivastava A. Effect of polysorbate 80 concentration on thermal and photostability of a monoclonal antibody. AAPS PharmSciTech. 2013; 14:1-9.
14. Kröber T, Knöchlein A, Eisold K, Kalbfuβ-Zimmermann B, Reichl U. Dna depletion by precipitation in the purification of cell culture-derived influenza vaccines. Chem Eng Technol. 2010; 33:941-59.
15. Rodrigues G A, Shalaev E, Karami T K, Cunningham J, Slater N K H, Rivers H M. Pharmaceutical development of AAV-based gene therapy products for the eye. Pharm Res. Pharmaceutical Research; 2019; 36:1-20.
16. Suomela H. Inactivation of viruses in blood and plasma products. Transfus Med Rev. Elsevier, 1993; 7:42-57.
17. Pamphilon D. Viral inactivation of fresh frozen plasma. Br J Haematol. 2000; 109:680-93.
18. FDA. Q5A Guidance Document: Viral Safety Evaluation of Biotechnology Products Derived from Cell Lines of Human or Animal Origin. Fed Regist. 1998; 63:51074-804.
19. Coulter B. Vi-Cell Reference Manual. 2011.
20. ASTM International. Standard Test Method for Foaming Properties of Surface-Active Agents. 2007;
21. Qiao C, Koo T, Li J, Xiao X, Dickson J G. AAV capsid structure and cell interactions. Gene Ther. 2011.
22. Rayaprolu V, Kruse S, Kant R, Venkatakrishnan B, Movahed N, Brooke D, et al. Comparative analysis of adeno-associated virus capsid stability and dynamics. J Virol. 2013; 87:13150-60.
23. Rego M, Hanley L M, Ersing I, Guerin K, Tasissa M, Haery L, et al. Improved yield of AAV2 and rAAV2-retro serotypes following sugar supplementation during the viral production phase. bioRxiv. 2018; 1-21.

1. A membrane rupture solution comprising:
one or more purified non-ionic detergents, wherein the non-ionic detergent is selected from the group consisting of ethoxylated alcohol, sorbiton ethoxylate, triblock copolymer and combinations thereof, wherein the detergent is in a concentration range from about 0.01% (w/v) to about 2.0% (w/v); and
a scavenger, in a concentration in a range of 0% to about 2.0%.

2. The solution of claim 1, wherein the ethoxylated alcohol is TDA9, tergitol 15-S-9, or combinations thereof.

3. The solution of claim 1, wherein the sorbiton ethoxylate is polysorbate 20, polysorbate 80, or combinations thereof.

4. The solution of claim 1, wherein the triblock copolymer is poloxamer 188, poloxamer 407, poloxamer 305, or combinations thereof.

5. The solution of claim 1, wherein the non-ionic detergent is in a concentration of about 0.25% (w/v).

6. The solution of claim 1, wherein the non-ionic detergent is TDA9 in a concentration of about 0.05% (w/v) and polysorbate 20 in a concentration of about 0.2% (w/v).

7. The solution of claim 1, wherein the scavenger is selected from the group consisting of ethylenediaminetetraacetic acid (EDTA), ethanol, deoxythymidine triphosphate (DTTP), polyethylenimine (PEI), a PEI derivative, and combinations thereof.

8. The solution of claim 1, wherein the non-ionic detergent comprises ethoxylated alcohol at a concentration of about 0.05% (w/v), polysorbate 20 at a concentration of about 0.2% (w/v) and poloxamer 188 at a concentration of about 0.005% (w/v).

9. The solution of claim 8, further comprising the scavenger is PEI at a concentration of about 0.1% (w/v).

10. The solution of claim 1, further comprising a stabilizer.

11. The solution of claim 10 wherein the stabilizer is selected from the group consisting of sucrose, magnesium chloride, sodium chloride, PEG of various chain lengths, and combinations thereof.

12. A method of extracting intracellular material from cells comprising contacting the cells with the membrane rupture solution according to claim 1, wherein the cells are lysed.

13. The method according to claim 12 wherein the intracellular material is viral vector, protein and/or peptide.

14. The method according to claim 12 wherein the cells are mammalian or insect cells.

15. The method according to claim 12 wherein the solution is directly added to the cells in cell culture media or to a cell suspension, and wherein the cells are then centrifuged to remove cell media and resuspended in a suitable buffer.

16. The method of claim 15 wherein the buffer is selected from the group consisting of Tris, phosphate, histidine, citrate, acetate, and combinations thereof.

17. The method of claim 12, wherein the scavenger is added only after cell lysis.

18. A method of inactivating viruses comprising contacting a biological product with the membrane rupture solution according to claim 1.

19. The method according to claim 18 wherein the biological product contains at least one enveloped virus.

20. The method according to claim 18 wherein the biological product is exposed to the membrane rupture solution for about 1 minute to about 120 minutes at 2° C. to 40° C.

21. A method of purifying TDA9 to reduce product-related impurities, comprising:
(1) heating raw material to 50-180° C. while mixing with an inert gas or applying vacuum, wherein the raw material comprises impurities and TDA9;
(2) cooling the material of step (1) to 15-40° C. while mixing with inert gas;
(3) adding 10-50 volume percent of water to the material of step (2) to form a homologized solution;
(4) heating the solution of step (3) to 40-120° C. to complete hydrolysis of volatile impurities; and
(5) filtering the solution of step (4) with a 0.2-1.0 μm filter,
wherein the TDA9 is purified.

22. The method of claim 21 wherein the impurity comprises ethylene oxide and/or polyethylene glycol.
